(19) United States
(12) Patent Application Publication
(10) Pub. No.: US 2022/0370292 A1
(43) Pub. Date: Nov. 24, 2022

(54) AUTOMATED SYRINGE FILLING AND MEASURING SYSTEM AND METHODS OF USING SAME

(71) Applicant: Auto Vaccine Innovation, San Jose, CA (US)

(72) Inventors: Akhil Seth, San Jose, CA (US); Ian Coll McEachern, San Francisco, CA (US); Allen Jameson, Sunnyvale, CA (US)

(73) Assignee: Auto Vaccine Inovations, San Jose, CA (US)

(21) Appl. No.: 17/303,141

(22) Filed: May 21, 2021

(51) Int. Cl. A61J 1/20; A61J 1/14

(52) U.S. Cl. CPC A61J 1/2096; A61J 1/1406; A61J 2200/74; A61J 2205/10

(57) ABSTRACT

Methods and devices are provided precision filling of medical syringes. Generally, the methods and devices are designed to determine and measure dosages and fill a syringe with a precise amount from a medication vial and to store or transmit to a medical records storage system data associated with the process. In one embodiment, the fill apparatus weighs, photographs and stores information on a syringe and medicine vial, fills the syringe from the vial and confirms transfer of the medicine to the syringe from the vial.

### CROSS-REFERENCE TO RELATED APPLICATION

This application claims priority to and the benefit of U.S. Provisional Application Ser. No. 63/138,472 filed Jan. 2, 2021, which is incorporated by reference herein in its entirety.

### FIELD OF THE INVENTION

The present invention relates to devices and methods for filling of syringes and in particular to improved methods to precisely fill syringes with medications and/or vaccines and to record and store information related to the filling of the syringe.

### BACKGROUND

Syringes for the administration of medicines in the medical field as well as in other endeavors are in common use. While there exist prefilled syringes, a majority of medication syringes used today are filled at the point of use. Filling at the point of use provides a number of advantages in inventory, storage and shelf life and cost as well as the ability to do patient specific formulations and dosages. While there are advantages to point of care filling of syringes, drawbacks also exist. In many point of care instances the filling of the syringe is a manual operation. This can lead to issues related to medication tracking, filling errors and waste. While some automated filling systems are available, they can be large, cumbersome and expensive.

Particularly in applications in which repeated injections must be administered, conventional fill operations are time-consuming and labor intensive, requiring close attention to the amount of fluid drawn into the chamber of the syringe as its plunger is retracted, as well as proper registration and/or coupling of the syringe to a fluid supply source in the first instance, and careful removal of the syringe from such fluid supply source after filling of the syringe. In instances in which multiple liquids are required, which must be mixed for administration, additional difficulties are present.

When such multiple liquids are involved, it is desired to mix them at the point of use in a manner that avoids degradation or impairment of liquid supplies, while at the same time achieving precision bulk filling of syringes, minimizing the incidence of needlestick events, minimizing the risk of errors, and increasing the efficiency and speed of preparation and subsequent injections.

In consequence, the art continues to seek improvements in syringe filling apparatus, and in associated processes and procedures

### SUMMARY

The present invention provides an apparatus and system for the automated filling of syringes. The system provides algorithms and the associated apparatus for accepting medication vials and syringes, recording and tracking pertinent information related to the vial and syringe, automatically filling the syringe with the a precise volume of medication in the vial and verifying the fill volume of the syringe.

According to a second embodiment of the invention, the automated syringe filling apparatus provides the algorithms and apparatus to prefill and pre-mix medication components in the medication vial prior to filling a syringe with the medication.

According to a third another embodiment of the present invention, algorithms and an associated apparatus are provided to transmit data obtained during the automated filling of a syringe to a hospital medical records system, an insurance medical records system and/or a cloud based medical records system.

The above-noted features and advantages of the present invention, as well as additional features and advantages, will be readily apparent to those skilled in the art upon reference to the following detailed description and the accompanying drawings, which include a disclosure of the best mode of making and using the invention presently contemplated.

The present disclosure relates generally to liquid fill and mixing apparatus and methods, and more specifically to syringe fill systems and methods, syringe fill system subassemblies that may be provided as disposables for single-use products, and syringes fillable by such syringe fill systems, subassemblies, and methods, as hereinafter more fully described.

In one aspect, the disclosure relates to a syringe fill system, comprising: a housing in which is mounted a syringe holding and actuating mechanism, a vial holding and movement mechanism, a vial and/or syringe weighing mechanism, a dosage input interface, a user interface for displaying information to the user and for accepting commands from the user, a camera for identifying and recording vial and syringe information, system memory, mechanical and control circuitry and a hospital record system interface, wherein the syringe pusher is coupled with a track in the housing for bidirectional linear movement and the vial is coupled with a track in the housing for bidirectional linear movement.

The system further comprises a syringe fixation housing for holding a medical syringe in a fixed location within the syringe filling system and a carriage and driver assembly wherein the carriage holds a medicine vial and wherein the carriage moves the medicine vial relative to the syringe such that the syringe needle punctures the medicine vial through the pierceable vial membrane and positions said syringe needle in a specified relationship to said vial membrane. Vials with pierceable membranes are disclosed in U.S. Pat. No. 10,618,700 titled MEDICAL VIAL CAP, the entirety of which is herein incorporated by reference.

The system further comprises a driver assembly operatively coupled with the pusher, arranged for user selection of an amount of a fluid to be dispensed and responsive to the user selection to translate the pusher distally toward the medicine vial and proximally away from the medicine vial to inject air into the vial and to draw the medicine in the vial into the syringe for dispensing to the user a precise selected amount of the medicine. The driver assembly also prevents reverse motion of the syringe plunger after drawing the medicine into the syringe while the syringe is located in the device.

A further aspect the disclosure relates to a method of filling a syringe with fluid at a point of use, such method comprising use of a syringe fill system or syringe fill system subassembly of the present disclosure.

A still further aspect of the disclosure relates to a therapeutic composition supply kit, comprising a syringe fill device according to the present disclosure, and at least two components A and B.

Component A is stored in a first vial and is a solid or a liquid. Component B is stored in a second vial and is a liquid. Components A and B are mixed to form a combination component and the mixing is done in the first vial at a predetermined ratio between components A and B. Mixing of the two components may be mixed outside of the syringe filling apparatus or the syringe filling apparatus may be used to facilitate the mixing of the two components.

Other aspects, features and embodiments of the disclosure will be more fully apparent from the ensuing description and appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 shows a first perspective view of a medical syringe filling apparatus;

FIG. 2 shows a second perspective view of the apparatus of FIG. 1;

FIG. 3 shows rear perspective view of the apparatus of FIG. 1;

FIG. 4 shows a perspective view of the medical syringe filling apparatus of FIG. 1 with a portion of the housing removed to provide visualization of a scale assembly;

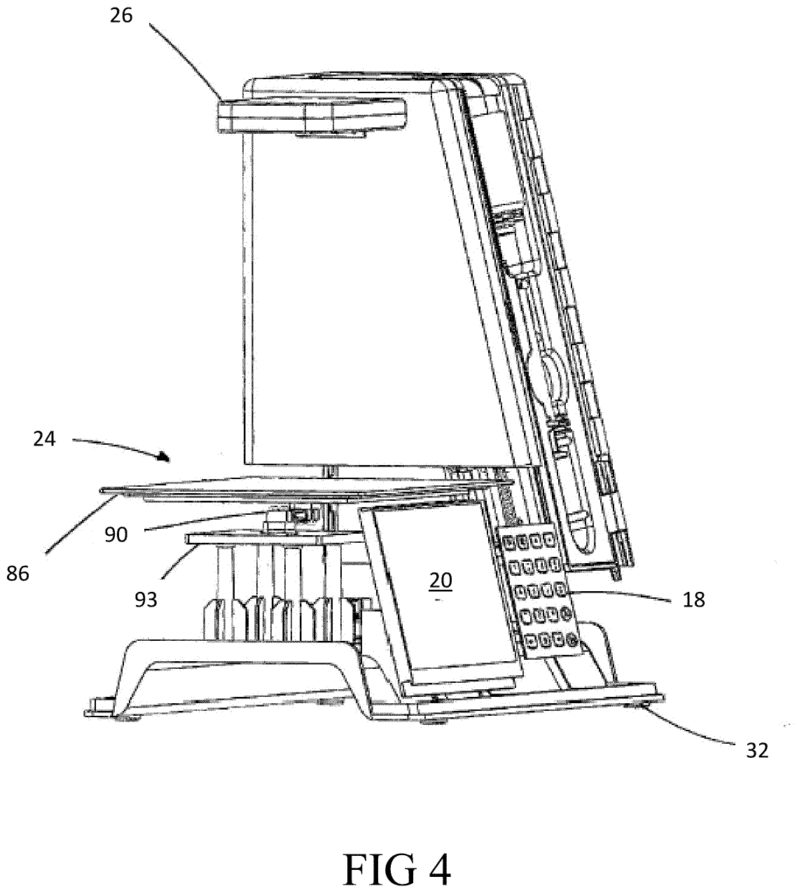

FIG. 5 shows an exploded view of the syringe carriage assembly and the vial carriage assembly of the apparatus of FIG. 1;

FIG. 6 shows a perspective view of the apparatus of FIG. 1 with the syringe and vial removed;

FIG. 7. Shows a front view of the apparatus of FIG. 1 with the front door removed;

FIG. 8 shows an exploded view of the imaging device of the apparatus of FIG. 1;

FIG. 9A shows a front view of the base and scale assembly of the apparatus of FIG. 1;

FIG. 9B shows a front view of the base and scale assembly of a first alternate embodiment of the scale assembly of the apparatus of FIG. 1;

FIG. 9C shows a front view of the base and scale assembly of a second alternate embodiment of the scale assembly of the apparatus of FIG. 1;

FIG. 10 shows a schematic of the electronic assembly of the apparatus of FIG. 1;

FIG. 11 shows a flowchart of a method for filling a syringe using the apparatus of FIG. 1;

FIG. 12 shows an alternative embodiment of the plunger and vial motor assemblies of the apparatus of FIG. 1;

FIG. 13 shows a rear view of the recess housing of the apparatus of FIG. 1 including temperature and presence sensors;

FIG. 14 shows a side section view of the recess housing of the apparatus of FIG. 1;

FIG. 15 shows a view of the vial carriage assembly the apparatus of FIG. 1 including a motor for vial rotation;

FIG. 16 shows a front view of the design of the apparatus of FIG. 1;

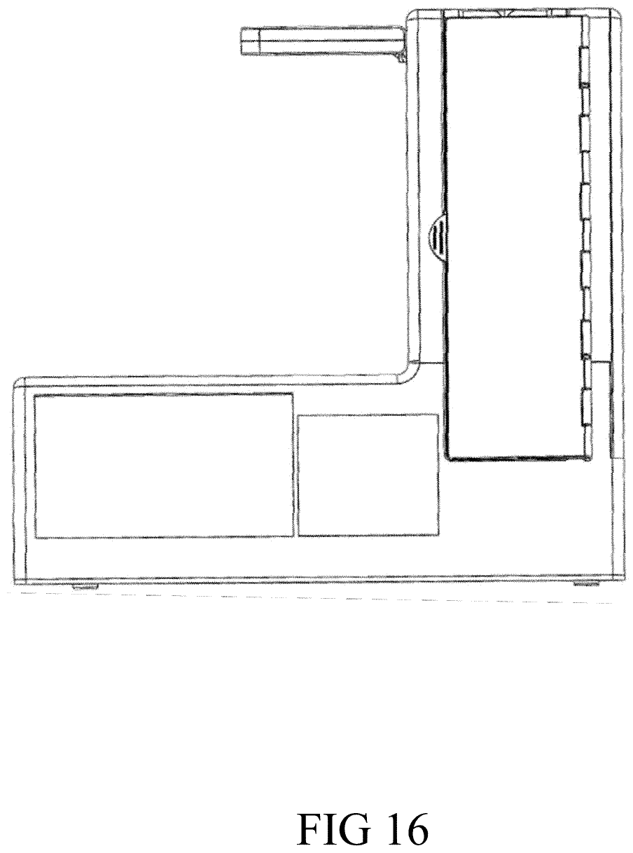

FIG. 17 shows a rear view of the design of the apparatus of FIG. 1;

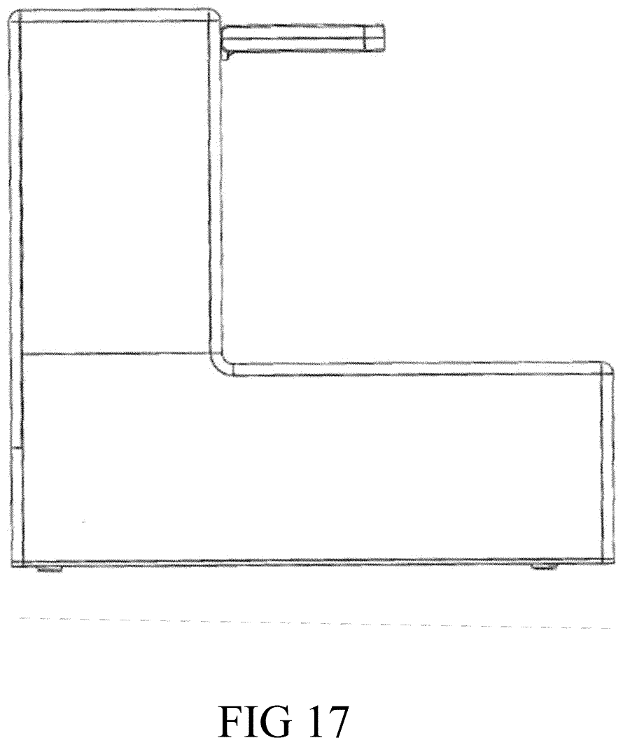

FIG. 18 shows a right side view of the design of the apparatus of FIG. 1;

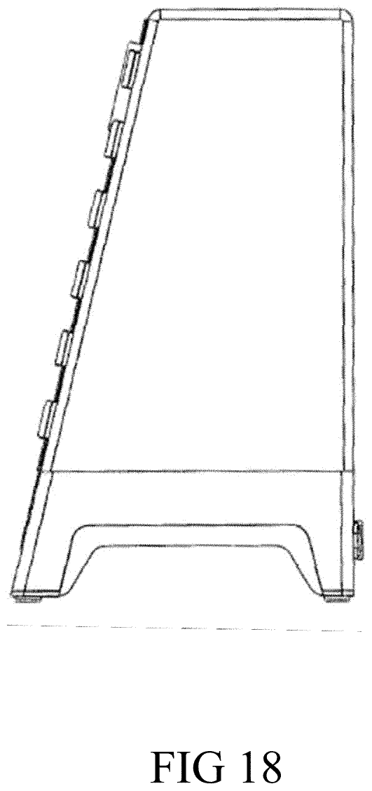

FIG. 19 shows a left side view of the design of the apparatus of FIG. 1;

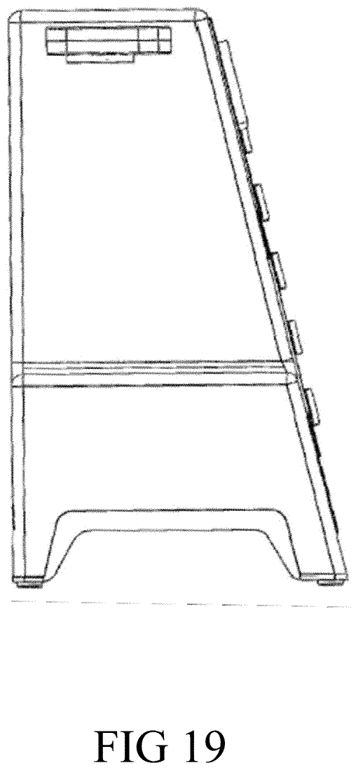

FIG. 20 shows a top view of the design of the apparatus of FIG. 1;

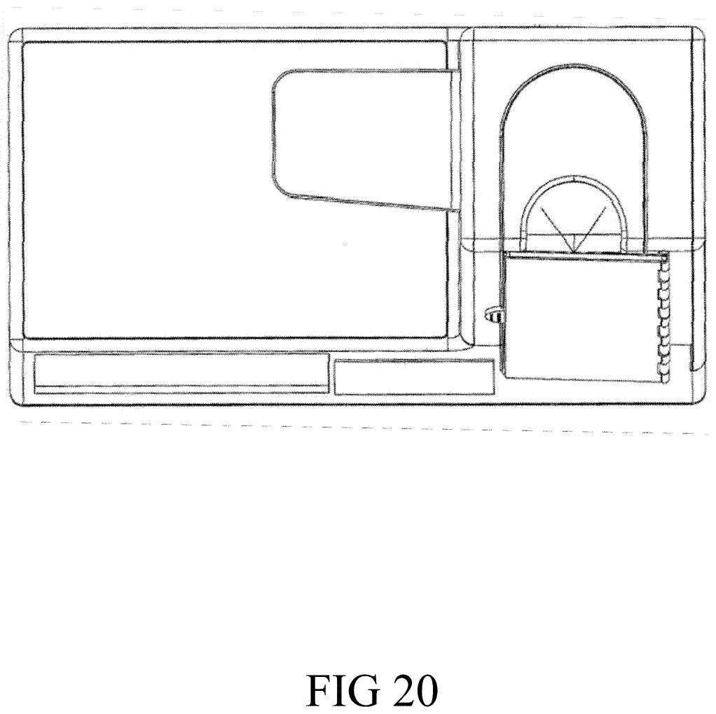

FIG. 21 shows a bottom view of the design of the apparatus of FIG. 1;

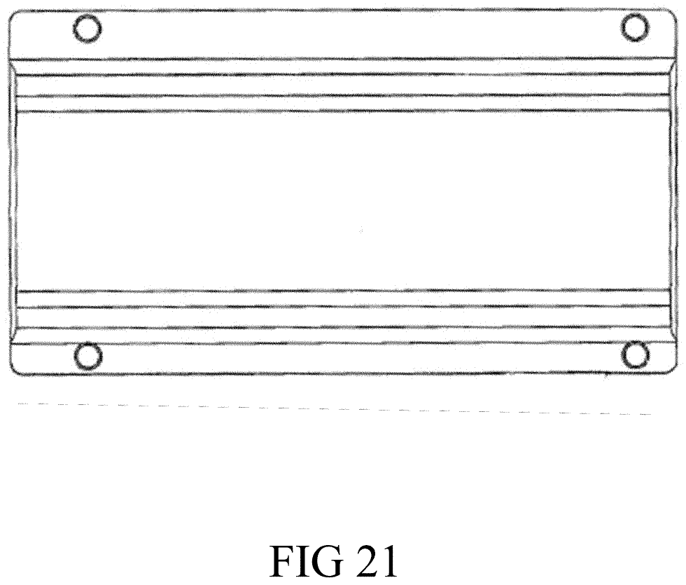

FIG. 22 shows a perspective view of the apparatus of FIG. 1;

FIG. 23 shows a top view of the apparatus of FIG. 1 with a closed door;

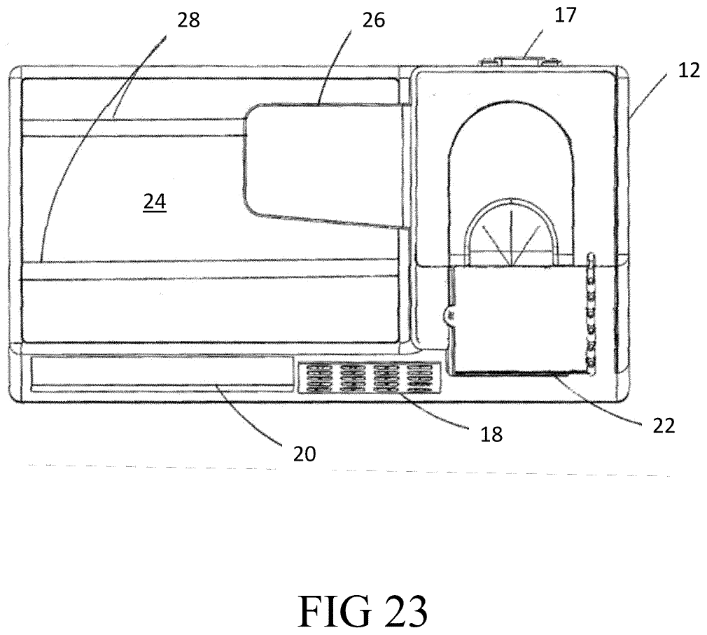

### DETAILED DESCRIPTION

The present disclosure relates generally to liquid fill and mixing apparatus and methods, and more specifically relates to syringe fill systems and methods.

It will be recognized that the scope of the present disclosure is not limited by specific applications, and that the apparatus and methods described herein may be employed in a variety of implementations, e.g., in orthopedics, physical therapy, dentistry, dermatology, cosmetic surgery, and numerous other fields. Further, while the ensuing disclosure includes illustrative embodiments employing mechanisms for precisely filling syringes from a medicine vial, recording medicine and syringe information, storing and transmitting data to a medical records system and delivery and mixing of two separate fluids from respective source containers, it will be appreciated that the disclosure contemplates mixing of multiple components as well as various means to mix, purge weigh and record data related to the filling of the syringe.

The present disclosure contemplates syringe fill systems, subassemblies of such systems, syringes that can be filled using such fill systems or subassemblies, kits, and related methods.

To precisely fill a syringe, a medicine vial containing a liquid form of medicine is first placed on the weighing platform of the filling apparatus and the weight of the vial with medicine is recorded by the system and the vial is photographed to record the label information on the vial. As part of this process the camera system may include a bar code reader a text identifying reader or other types of readers known in the art to specifically read and record information related to the medicine vial. The vial is then placed in the vial carriage on the apparatus. Next, a syringe is place on the weighing platform of the apparatus and the weight of the syringe is recorded. The syringe may be photographed, and the camera may do recognition of information on the syringe as was done with the vial above. The syringe is then placed in the syringe holder portion of the apparatus. The user inputs the requested fill dosage for the syringe into the user input console of the apparatus and the apparatus causes the plunger of the syringe to mover from a first position where the syringe plunger is against the distal end of the syringe cylinder and a second position where the syringe cylinder is retracted to pull an amount of air into the syringe equal to the dosage requested. Next the vial carriage moves the vial from a first position where the vial is distal to the distal end of the syringe needle to a second position where the syringe needle punctures the pierceable membrane of the vial. Next, the plunger carriage portion of the apparatus moves the plunger from the second position back to the first position to push the air in the syringe into the vial to offset the volume of fluid to be extracted for the medicine dose. The syringe plunger is then moved from the first position to a third position where the syringe plunger is retracted an amount sufficient to precisely fill the syringe with the selected medicine dosage. The vial carriage next moves the vial from its second position back to its first position such that the syringe needle disengages the pierceable membrane of the vial. A UV light then sterilizes the needle and the exterior of the pierceable membrane of the medicine vial. The user then removes the syringe from the plunger actuation portion of the apparatus and reweights the plunger to record the change in weight between the empty syringe and the filled syringe. The user next removes the vial from the vial carriage and weights it a second time to compare the weight of the vail after syringe filling to that prior to syringe filling. Also included in the apparatus is a user information screen that displays weights, medicine information, steps for completing the syringe filling and any potential errors in the system.

The syringe fill system of the disclosure may be adapted for manual operation, automated operation, or a combination of manual and automatic operation.

The syringe fill system subassembly may also be provided as a disposable unit, e.g., fabricated predominately of plastic and elastomeric material(s) of construction, or otherwise constructed and arranged for disposable or multiple use. Further, the plastic and elastomeric materials may be sterilizable by various methods such as detergents, ethylene oxide gas, radiation or other cleaning and sterilization methods know in the art.

Syringes useful in the broad practice of the present disclosure include syringes comprising a barrel defining an interior volume that is adapted for filling with a fluid. The syringe may be empty prior to using the filling apparatus or the syringe may include in its interior volume a solid material, e.g., a particulate solid material, that is solubilized or suspended when the syringe is filled with the fluid, to constitute a composition for administration by the syringe. The solid material may comprise a buffering agent such as sodium bicarbonate, or other solid material useful to make up a fluid formulation for subsequent administration by the syringe.

In another aspect, the disclosure contemplates a method of filling a syringe with fluid at a point of use, in which the method comprises use of an above-described syringe fill system or syringe fill system subassembly. Such method may be carried out with a medicine such as a vaccine.

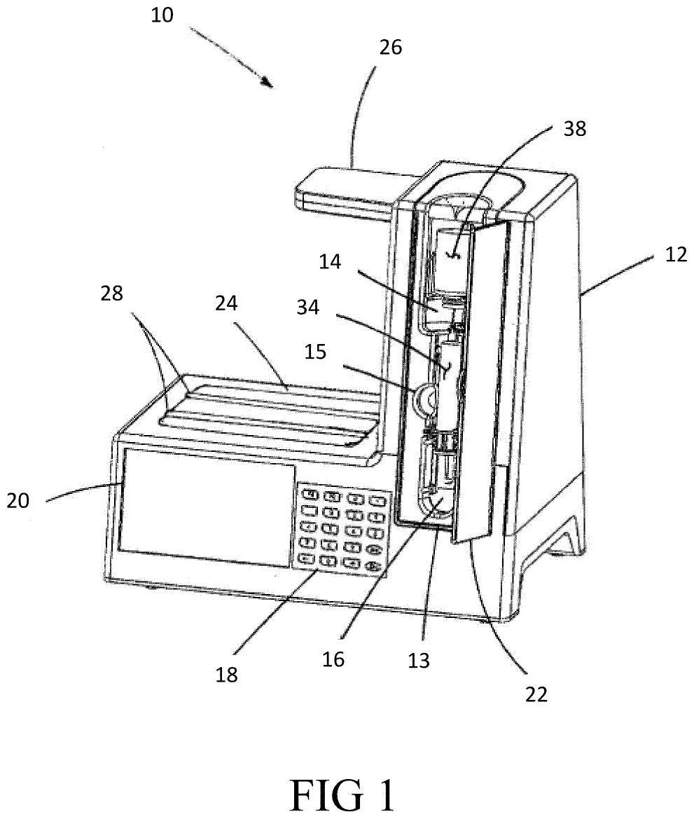

Referring now to the drawings, FIG. 1 is a perspective view of syringe fill apparatus 10 according to one embodiment of the disclosure.

The fill apparatus 10 includes housing 12. The front face of the housing 12 includes a vial recess 14 and a syringe recess 16. Vial recess 14 and syringe recess 16 may combine to form a single recess. The front face of the housing 12 also may include a keypad 18 for entering information and display 20 for communicating information to a user. In some embodiments, the display screen 20 may be a touch screen that incorporates the functions of the keypad 18. Housing 12 may also include a door 22 that is moveable to cover vial recess 14 and syringe recess 16.

Fill apparatus 10 also includes a scale 24 disposed to weigh, for example, syringes and medicine vials. Scale 24 may include ridges 28 on its surface to provide means to restrain cylindrical objects from rolling off of the scale 24. Fill apparatus 10 further includes an imaging device 26 disposed to capture images of objects on the scale 24.

At its rear portion, the housing 12 includes a removable rear panel 30 and/or side panel 31, permitting access to internal components in the interior volume of the housing. The housing 12 is provided on its bottom face with bumper feet 32, which may be made of rubber or other elastomer material or may be made of metal, plastic or other hard materials. Bumper feet may also include springs (not shown) to damp out vibrations transmitted from the surface on which fill apparatus 10 is placed.

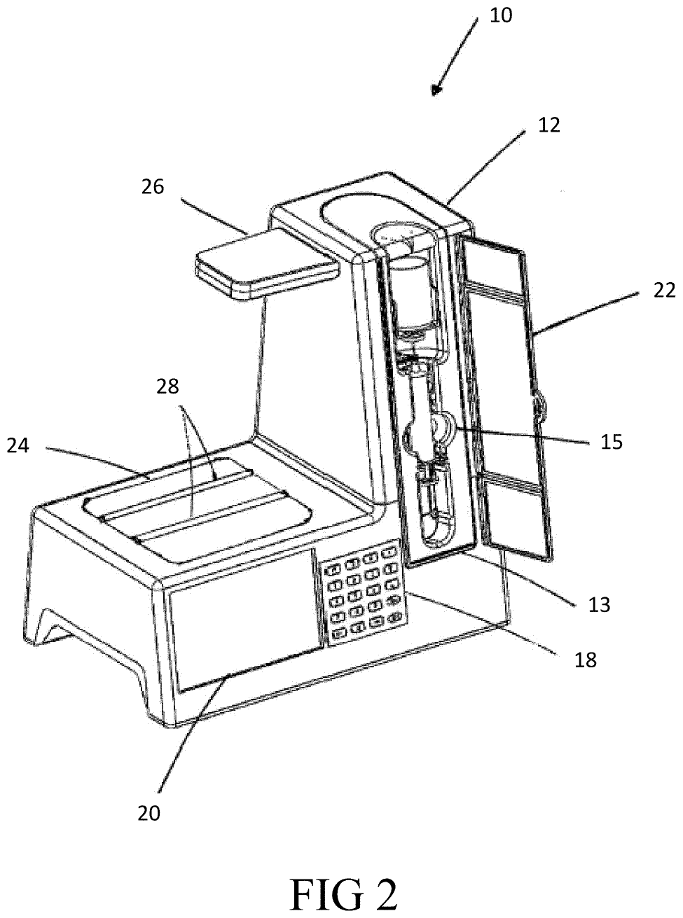

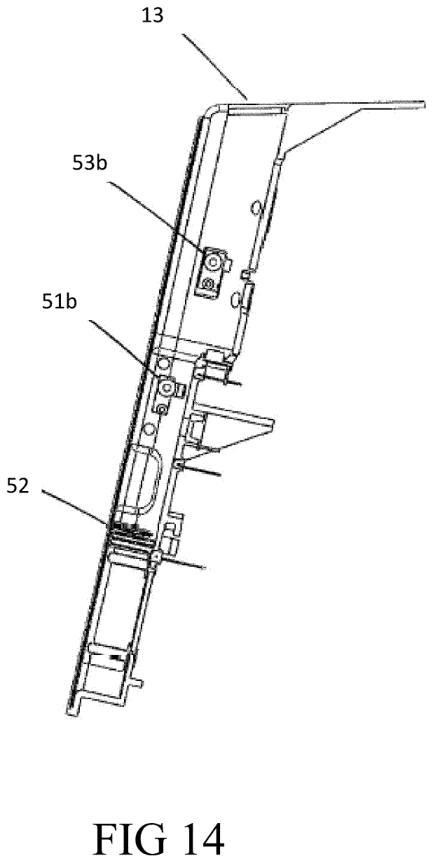

FIG. 2 is a second perspective view of the syringe fill apparatus of FIG. 1, coupled with a medicine vial and a syringe for filling thereof. As illustrated, a syringe 34 is shown as being coupled with the syringe coupling 36 in the syringe recess 16 of the fill apparatus 10. The syringe includes a cylinder portion 42 and a plunger 44. Syringe recess 16 includes a plunger carriage 46 and a static cylinder fixator 48. Static cylinder fixator 48 is disposed to interact with cylinder tabs 50 of cylinder portion 42 to hold cylinder portion 42 stationary in relation to the remainder of fill apparatus 10 while plunger 44 is moved relative to cylinder portion 42 during the filling of syringe 34. One or both of cylinder fixator 48 and plunger carriage 46 may be designed with a slight interference fit to their respective interfaces on the syringe 34. In a second embodiment one or both of cylinder fixator 48 and plunger carriage 46 may be designed with spring clips 52 (shown in FIG. 14) such that spring clips 52 frictionally engage the respective interfaces on the syringe 34 when the syringe 34 is placed in fill apparatus 10. In a third embodiment clamp mechanisms (not shown) disposed in the plunger carriage 46 and the cylinder fixator 48 may be designed to engage their respective interfaces on the syringe 34. The clamp mechanisms may be actuated manually or may be automated through use of solenoids, gears or other fixation mechanisms known in the art to allow the fill apparatus 10 to grip the interfaces of various sizes of plunger 44.

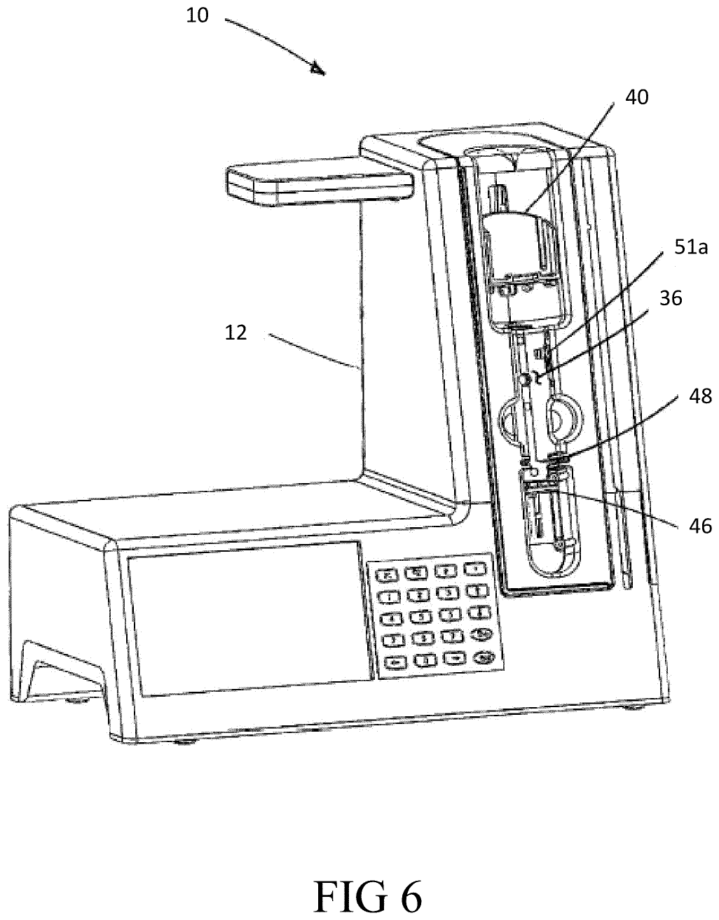

A vial 38 is shown as being coupled to a vial carriage 40 in vial recess 14 of the fill apparatus 10. Vial carriage 40 is disposed to engage with the vial neck 54 of vial 38. Vial carriage 40 may be designed with a slight interference fit to vial neck 54. In a second embodiment vial carriage 40 may be designed with spring clips 52 (shown in FIG. 6) such that spring clips 52 frictionally engage the vial neck 54 when the vial 38 is placed in fill apparatus 10. In a third embodiment clamp mechanisms (not shown) disposed in the vial carriage 40 may be designed to engage the vial neck 54. The clamp mechanisms may be actuated manually or may be automated through use of solenoids, gears or other fixation mechanisms known in the art.

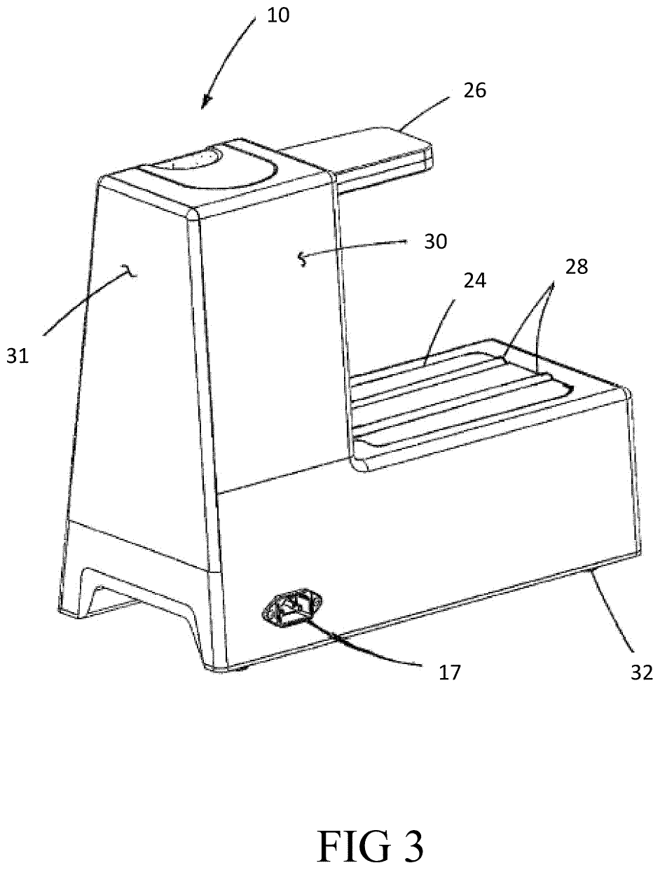

FIG. 3 is a rear perspective view of fill apparatus 10 of FIG. 1 and FIG. 2, showing, in addition to some of the components in FIG. 1 and FIG. 2, bumper feet 32 and electrical connector 17. Electrical connector 17 is disposed to allow for the attachment of a standard electrical cord for supplying power to the fill apparatus 10. Bumper feet 32 are made of materials to provide stability of the fill apparatus 10 on a table or other support and are made of elastomeric or other materials know in the art to provide sufficient coefficient of friction to keep the fill apparatus 10 from sliding off of its supporting structure or may alternatively be made of hard materials such as plastic or metal as described above.

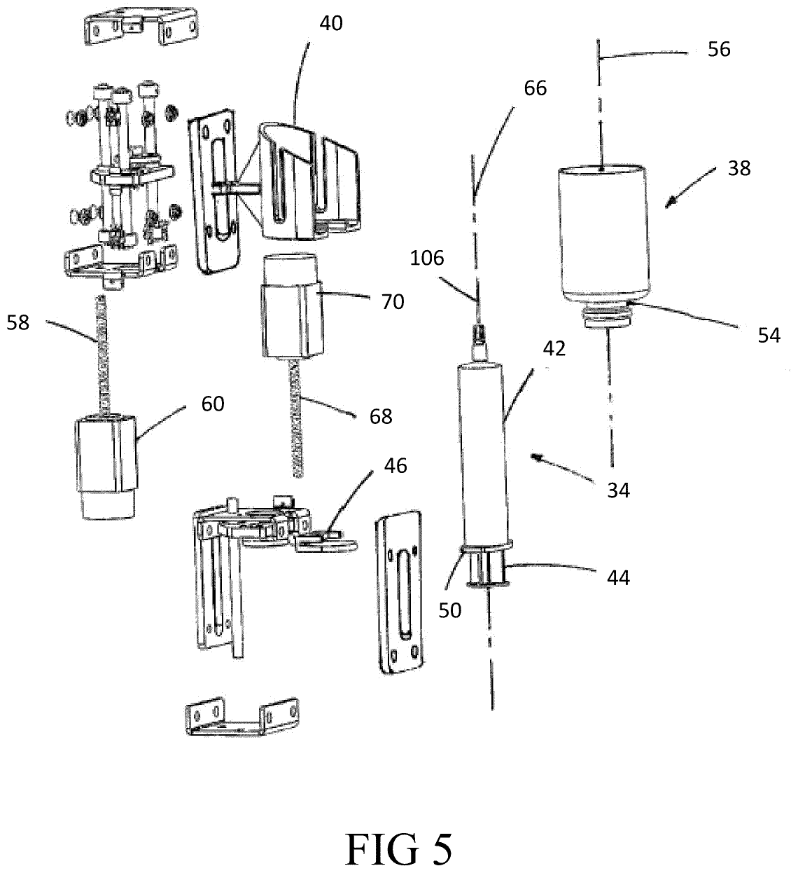

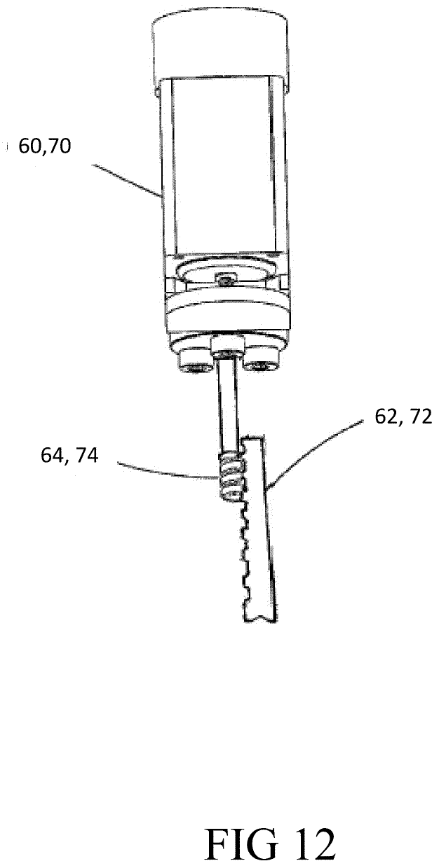

FIG. 5 is an exploded detail of the vial and plunger movement assemblies of the fill apparatus 10. As illustrated in FIG. 1 and FIG. 5, vial carriage 40 is disposed in vial recess 14 for linear motion along a vial axis 56. Vial carriage 40 is connected to vial lead screw 58 which is connected to vial motor 60. Vial motor 60 may be a dc motor, a stepper motor an ultrasonic motor or any rotary motor known in the art. Rotation of vial motor 60 causes vial lead screw 58 to rotate which in turn causes vial carriage 40 to move linearly along vial axis 56. In an alternative embodiment, the vial motor 60 and the vial lead screw 58 are replaced by a solenoid type linear actuator (not shown). In yet another embodiment the vial lead screw is replaced by a vial gear rack 62 which is driven by vial worm gear 64 which is in turn driven by vial motor 60 as shown in FIG. 12.

As further illustrated in FIG. 1 and FIG. 5, plunger carriage 46 is disposed in syringe recess 16 for linear motion along a syringe axis 66. Plunger carriage 46 is connected to plunger lead screw 68 which is in turn connected to plunger motor 70. Plunger motor 70 may be a dc motor, a stepper motor, an ultrasonic motor or any rotary motor known in the art. Rotation of plunger motor 70 causes plunger lead screw 68 to rotate which in turn causes plunger carriage 46 to move linearly along syringe axis 66. Syringe axis 66 and vial axis 56 may be disposed on the same axis or may be laterally offset from one another. In an alternative embodiment, the plunger motor 70 and the plunger lead screw 68 are replaced by a solenoid type linear actuator (not shown). In yet another embodiment the vial lead screw is replaced by a plunger gear rack 72 which is driven by plunger worm gear 74 which is in turn driven by plunger motor 70 as shown in FIG. 12

The plunger motor 70 and the vial motor 60 include encoders (not shown) for control of the motion of these motors. By determining the rotary motion of these motors through encoders, the linear motion of the plunger carriage 46 and the vial carriage 40 can be determined with a high level of accuracy. In another embodiment linear encoders (not shown) are placed in conjunction with the plunger carriage 46 and the vial carriage 40 to directly determine the location of these components relative to each other and to the housing 12.

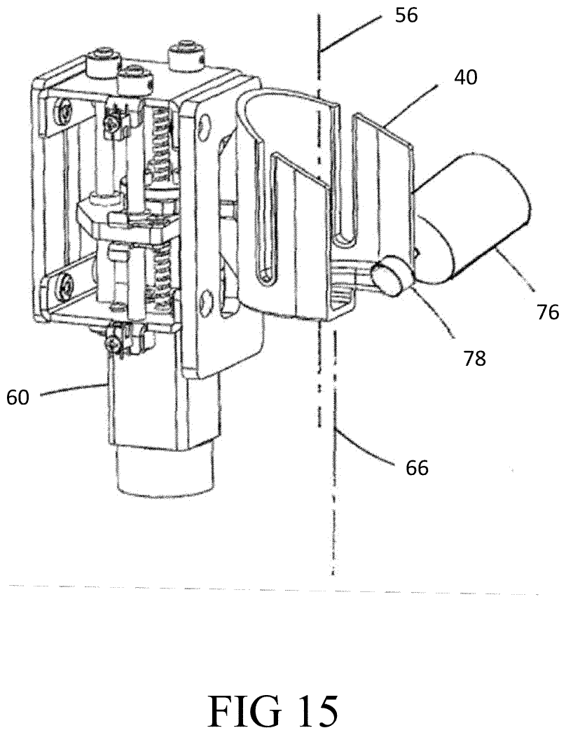

As further shown in FIG. 15, vial axis 56 may be laterally offset from syringe axis 66. Vial carriage 40 may include a vial rotation motor 76. Vial rotation motor 76 is attached to a vial rotation wheel 78 which frictionally engages the vial 38. After filling a syringe 34, vial rotation motor 76 causes vial rotation wheel 78 to rotate vial 38 a fractional portion of a full rotation such that subsequent punctures of the pierceable vial membrane 80 are offset from each other.

Vial carriage 40 also includes vial temperature sensor 82 for measuring the temperature of vial 38. Syringe recess 16 includes a syringe temperature sensor 84 for measuring the temperature of the syringe 34. Temperature sensors 82 and 84 may be contact temperature sensors, infrared temperature sensors or other temperature sensors know in the art. The fill apparatus 10 may have logic that prevents dispensing of medicine from the vial 38 until the vial 38 is within a specific temperature range. In addition, the fill apparatus 10 may include means to prevent removal of the syringe 34 unless the syringe is within a specified temperature range. In alternative embodiments, the fill apparatus may also include heating or cooling elements (not shown) in one or both of the vial carriage 40 and the syringe recess 16 for maintaining the vial 38 and/or the syringe 34 within a specified temperature range.

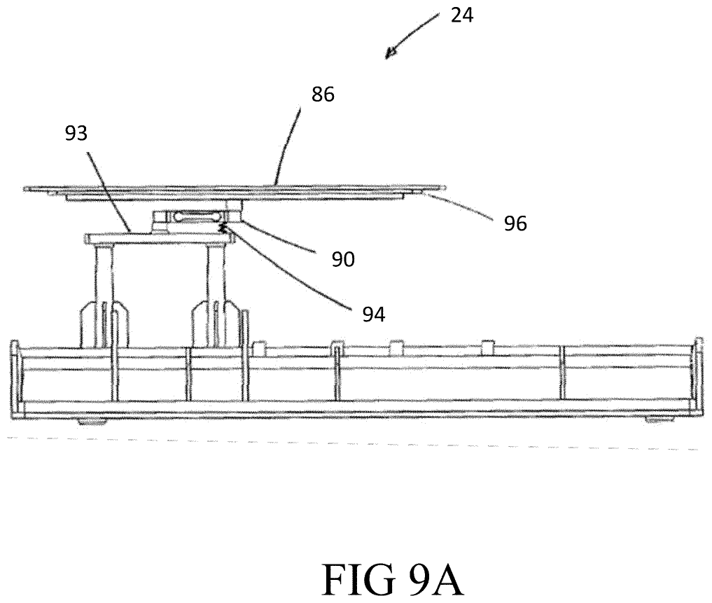

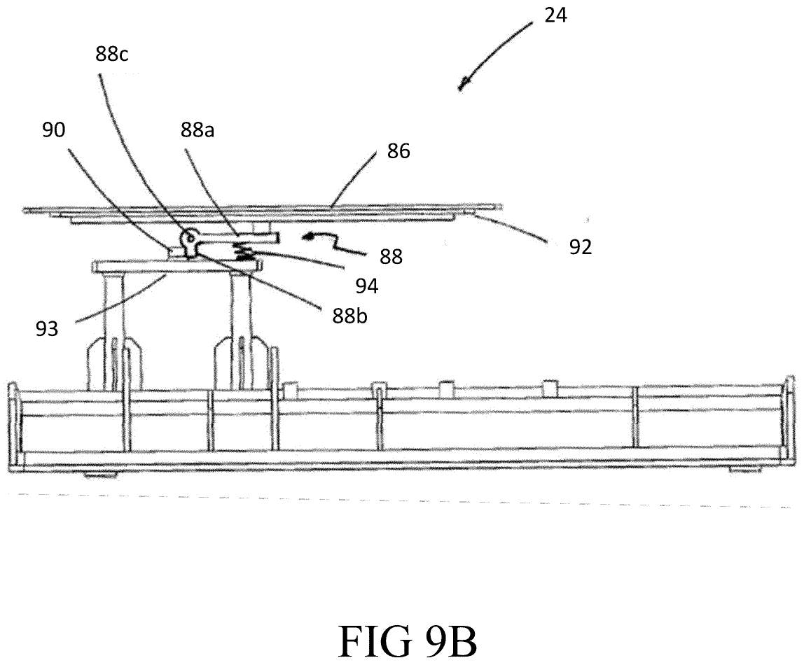

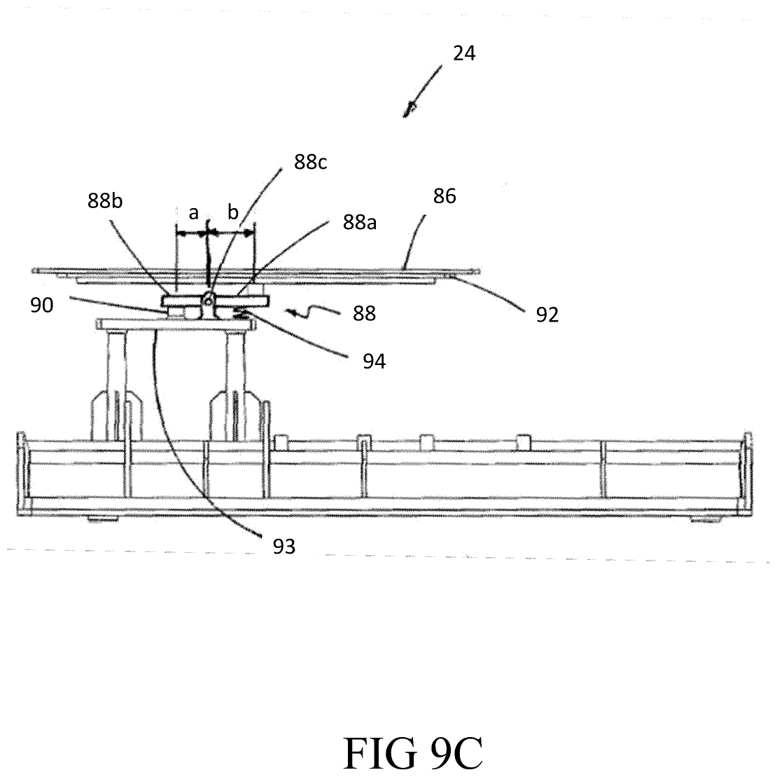

As shown in FIG. 9A-9C, scale 24 includes a scale plate 86, a scale lever 88, a scale pressure sensor 90 and a scale temperature sensor 92. Scale plate 86 may include ridges 28 such that items placed on the scale plate 86 are constrained to prevent them from rolling off of the scale plate 86. Scale lever 88 has a first lever arm 88a and a second lever arm 88b and a pivot 88c. First lever arm 88a is connected to scale plate 86 and second lever arm 88b is connected to scale pressure sensor 90. The orientation between first lever arm 88a and second lever arm 88b is used to change the orientation between the scale plate 86 and the scale pressure sensor 90. For instance, first lever arm 88a and second lever arm 88b can be aligned on the same axis. In this instance, the scale plate 86 will impart a vertical force to first lever arm 88a and scale pressure sensor 90 will measure a vertical force from second lever arm 88b. In a second instance, second lever arm 88b is oriented perpendicular to first lever arm 88a. In this instance, the scale plate 86 will impart a vertical force to first lever arm 88a and scale pressure sensor 90 will measure a horizontal force from second lever arm 88b. Additionally, the length ratio between first lever arm 88a and second lever arm 88b (computed as the ratio of dimension a and b) can be used to adjust the range and sensitivity of scale pressure sensor 90. For instance, if first lever arm 88a is longer than second lever arm 88b, then the force applied to the scale plate 86 is amplified at the scale pressure sensor 90 such that the overall range that of weights on the scale plate 86 is reduced and the weight sensitivity as measured by the change in scale pressure sensor 90 output in relation to a change in the weight applied to scale plate 86 is increased. Alternatively, if the second lever arm 88b is longer than the first lever arm 88a then the sensitivity of the scale pressure sensor 90 is reduced and its overall range is increased. In another embodiment, a scale spring 94 is attached to the scale lever 88 to offset the tare weight of the scale plate 86. As the scale pressure sensor 90 may be sensitive to temperature variations, a scale temperature sensor 92 is included to obtain the temperature of the scale pressure sensor 90 such that the output of the scale pressure sensor 90 can be adjusted for temperature variations, increasing the accuracy of the scale pressure sensor 90. Scale temperature sensor 92 may be a flat plat temperature sensor designed to make temperature readings across a wide area or may be a discrete temperature sensor design to measure the temperature at a point. If the scale temperature sensor 92 is discrete, it is placed in close proximity to the scale pressure sensor 90 so that the effect of temperature variation on the scale pressure sensor 90 is more accurately determined.

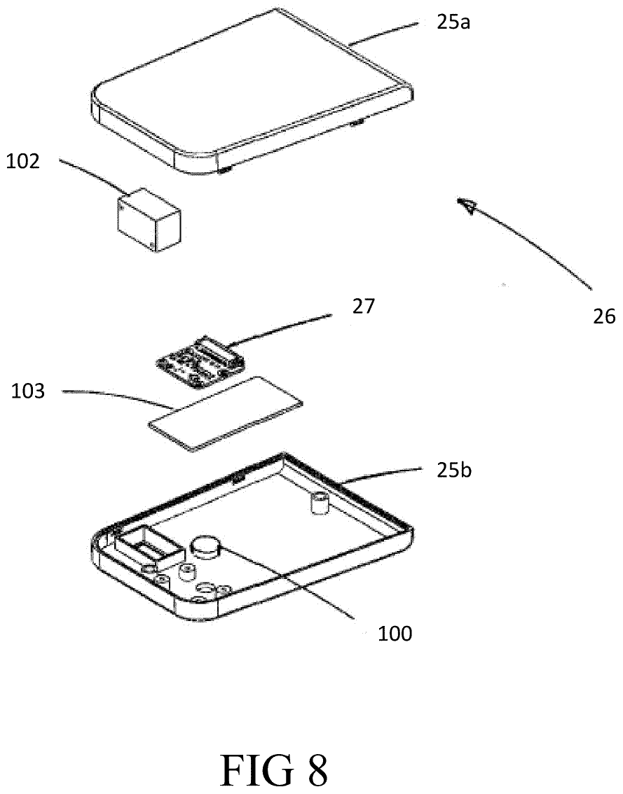

FIG. 8 is an exploded view of the imaging device 26. Imaging device 26 comprises a camera 27, a bar code scanner 102, a bar code scanner board 103, a lens system 100 and imaging housings 25a and 25b. The camera 27 and the bar code scanner 102 are disposed such that items placed on the scale 24 shown in FIG. 1 can be photographed and any bar codes on those objects read by bar code scanner 102. In an alternative embodiment, camera 27 contains an infrared temperature measurement system to enable the temperature of objects placed on scale 24 to be determined. In another embodiment, imaging device 26 includes a hinge (not shown) connecting the imaging device to the housing 12 of fill apparatus 10. In this embodiment, the imaging device 26 has a first position in which the imaging device 26 is rotated down against the housing 12 and a second position in which the imaging device is rotated up as shown in FIG. 1 such that its field of view is the scale 24. Rotation of the imaging device 26 from the first position to the second position engages a contact switch (not shown). Actuation of this contact switch may activate the fill apparatus 10 or may activate camera 27. Rotation of the imaging device 26 against the housing 12 may also protect the camera from damage when the device is not in use.

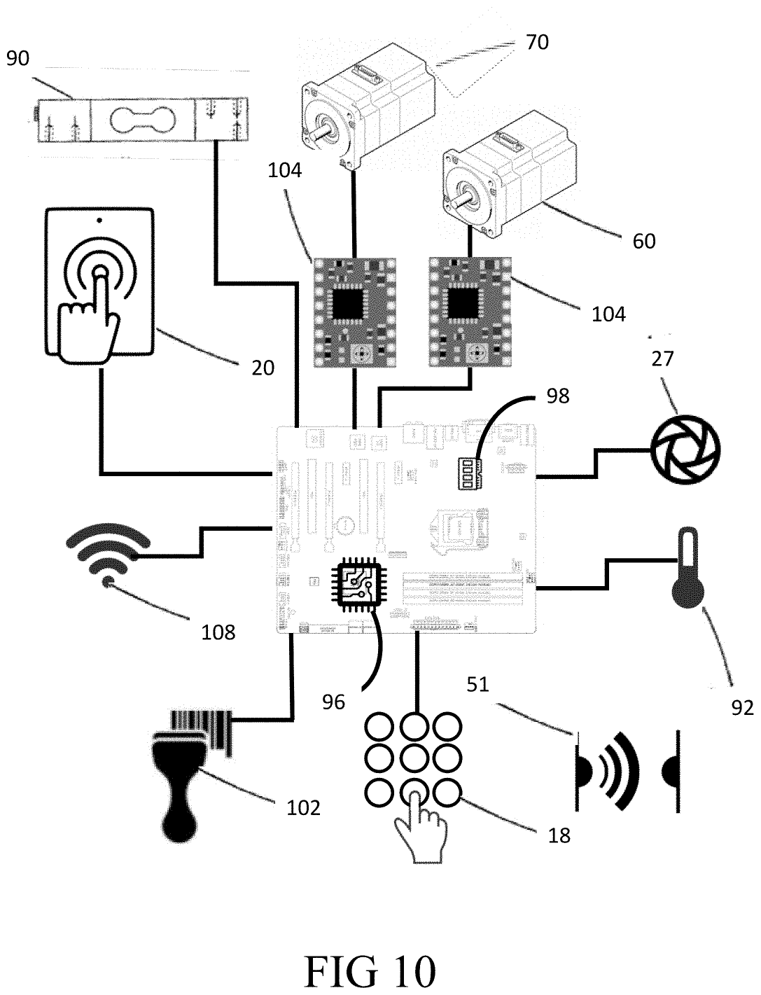

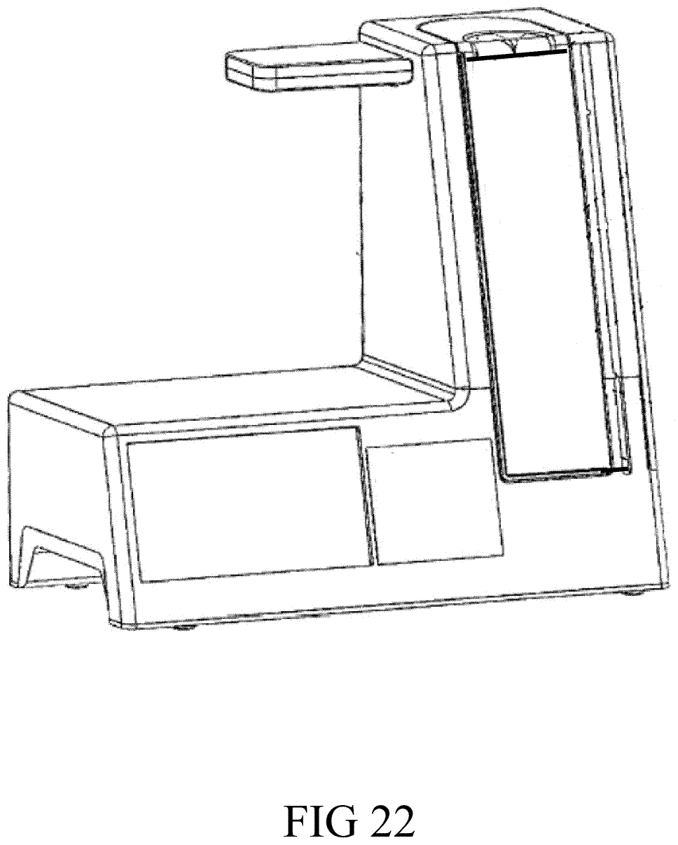

FIG. 22 is a plan view of scale plate 86 as seen from the imaging device 26. As previously disclosed, ridges 28 of scale plate 86 are placed to prevent objects placed on the scale plate 86 from rolling off. While two linear ridges 28 are shown, any suitable number of ridges 28 are contemplated. In addition, a single or multiple circular or ellipsoidal ridges may also be employed. Ridges 28 are also placed such that they indicate areas on the scale plate 86 to place specific items so that they can be imaged by the imaging device 26. Icons or indicators may be embossed, debossed or printed on the scale plate 86. Imaging device 26 can image items placed on scale plate 86. The fill apparatus 10 includes a processing unit 96 that takes the images obtained by the imaging device and stores the images in computer readable memory 98. The processing unit 96 (shown in FIG. 10) may also read QR codes on the items placed on the scale plate 86 and/or may perform text recognition of the information on the items placed on the scale plate 86 or bar code scanner board 103 may perform this function and transmit the information to the processing unit 96. This information may also be stored in computer readable memory 98. In an alternative embodiment, imaging device 26 is placed adjacent the syringe recess 16 and captures images of items placed on the scale plate 86. However as opposed to the first embodiment where the imaging device 26 views downward onto the scale plate 86, the imaging device 26 looks across the scale plate 86. This permits setting a vial on its base on the scale plate 86 and still being able to directly image the information contained on the vial label. In another embodiment, a mirror system may also be employed such that imaging device 26 looks downward onto both scale 24 and a mirror (not shown) wherein the mirror reflects an image of a field of view parallel to the top of the scale 24. In another embodiment, which is not shown, camera 27 is located inside of housing 12 and imaging device 26 comprises a set of mirrors (not shown) such that objects placed on scale plate 86 are reflected off of the mirrors and are imaged by the camera 27. In this embodiment the imaging device 26 may include a single mirror or multiple mirrors. If a single mirror is used the processing unit 96 reverses the image captured by camera 27 such that it appears as is the camera was imaging directly down on the scale plate 86. If multiple mirrors are used, they can be combined such that the image presented to the camera is a direct image and not a reflected image so that no additional processing of this image is required.

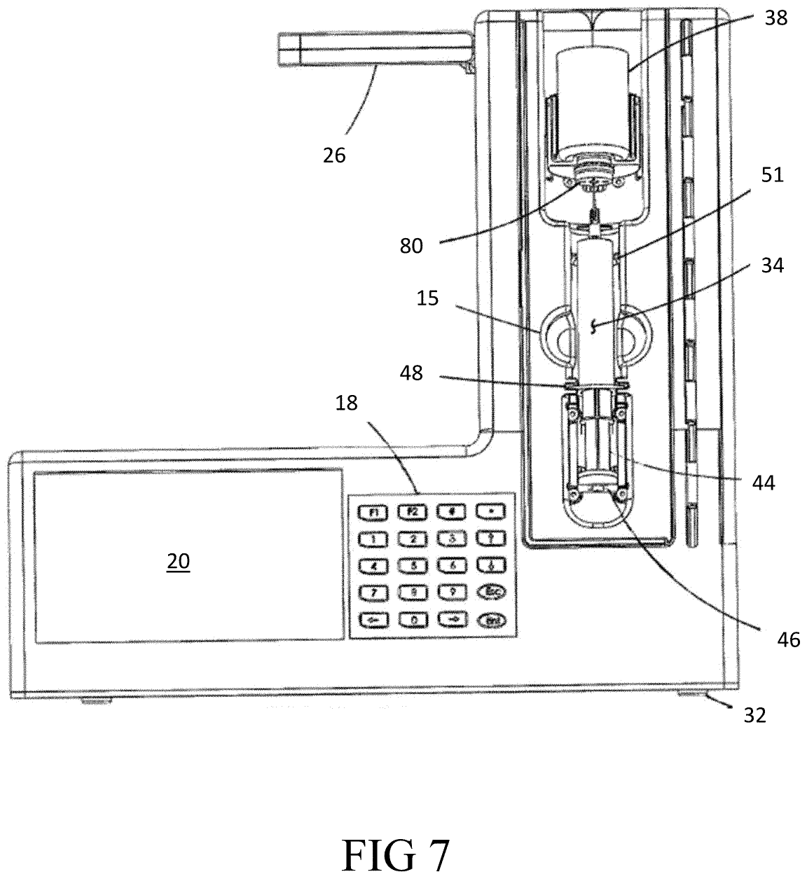

FIG. 7 shows a front view of the fill apparatus of FIG. 1. Located on the front of fill apparatus 10 is a keypad 18. They keypad 18 allows the user to enter information into the device. As described below, setup settings, dosages, medicine information, patient information and other data may be entered into the fill apparatus 10 using the keypad 18. The keypad 18 may also be used to allow the user to interact with and clear error screens that appear on the display screen 20. In addition, the keypad 18 may allow the user to directly input manual control of the moveable components of the fill apparatus 10 such as commanding the vial carriage 40 to move or may allow the user to activate specific components in the fill apparatus 10 such as activating the camera 27 to capture an image. Keypad 18 may also be kept in a sleep mode when the fill apparatus 10 is in the off condition such that it can sense the touch of a user to wake the system for use.

Also located on the front of fill apparatus 10 is a display screen 20. This screen is used to communicate information to the user. This information includes errors, steps for use, recommended actions, steps to clear errors as well as setup screens or other information. Display screen 20 may also show the images being captured by camera 27 such that the user may ensure that the items placed on the scale plate 86 have their information visible and readable. Display screen 20 may also communicate information to the user such as dosage recommendations or other similar information. Keypad 18 may provide a means to confirm information presented on display screen 20 such that the user is able to confirm that the image being recorded includes the appropriate information. The keyboard may also be used to request that the image be recorded a second time and that the original recorded image be deleted and replaced with the subsequent image.

The fill apparatus also contains a processing unit 96 (shown in FIG. 10) for controlling the motors, sensors, heaters, imaging, display and keying processes of the fill system 10. The processing unit 96 also has capabilities for making computations based on input and/or sensed data. The processing unit 96 can, for example take information on the medication and the patient such as patient weight, sex and age and automatically calculate a recommended dosage for dispensing into the syringe 34. The processing unit may be comprised of a single circuit board or of multiple interconnected circuit boards. In addition, the processing unit 96 may combine information from multiple sources into a single format. For instance, the processing unit 96 may add patient information such as name, weight, sex or other information to the image of the medicine vial 38, displaying the vial 38 image and the information in a single composite image.

Fill apparatus 10 may obtain and computer readable memory 98 may store information on medications, system functionality, system errors, system use, patient data, accessory data, user identification, settings and other similar information. The computer readable memory 98 may also store weights, images and similar information from each filling session. The computer readable memory 98 may also be configured to encrypt any information that it stores. As described above, the computer readable memory 98 is also capable of storing information related to the syringe filling, including but not limited to, the weights, the images, the dosages and the patient information related to each syringe fill. Alternatively, the syringe fill apparatus 10 may also include a memory slot (not shown) that can accept a removeable memory device (not shown). The memory slot may be disposed to accept a micro USB memory card, a flash memory card or other removable memory cards known in the art. The fill apparatus may also use the communications connector to store the information on an external hard drive or memory device connected via the communications connector.

The fill apparatus 10 also includes a communication module 108. The communication module interfaces with one or more of a hospital medical records system, a cloud-based records system, a patient database, a pharmacy database, a manufacturer database or other record systems known in the art. Communication is completed through one or more of an ethernet connection, a wireless connection such as a cellular network or a wi-fi network or other communications protocols as are known in the art. The communication module may pull information from said databases for use in preparing the medicine dosages described herein and may send information obtained during the dose filling process for storage in said databases. The communications module 108 may encrypt all communications that it sends and may have the ability to decrypt encrypted information sent to it. Communications module 108 may also include a direct communications connector (not shown). The communications connector may be a USB, RS323 or other style connector known in the art and may be disposed to allow accessories to be connected and to communicate with the fill apparatus 10. For instance, a patient scale (not shown) may be connected via the communication connector to directly transmit patient weight information to the fill apparatus 10. Communication port may also be used to power accessories devices through a USB connection as is known in the art.

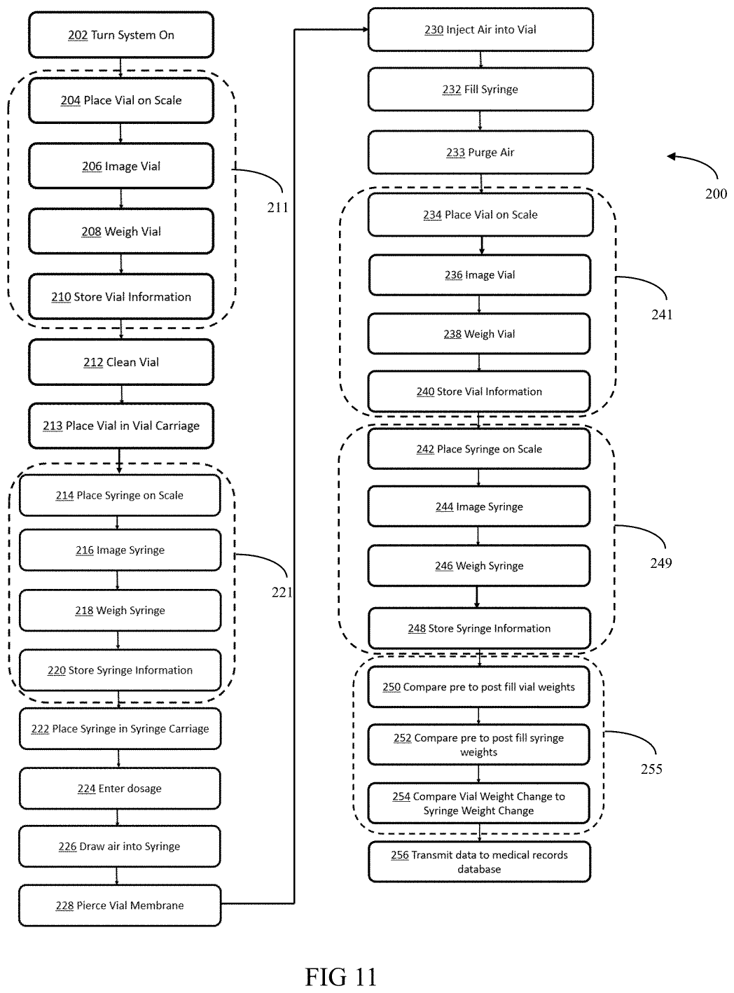

Referring now to FIG. 11, a process flow 200 for use of the fill apparatus 10 of FIG. 1 is shown.

In step 202, the fill apparatus is turned on.

In step 204, the user places a medicine vial 38 onto the scale plate 86 with the label of the vial 38 in a position for the imaging device 26 to capture an image of the vial 38.

In optional step 206 the processing unit 96 performs character recognition on the information contained on the vial label and reads any bar code information on the vial label.

In step 208 the user initiates a measurement of the weight of the vial 38. In this step the scale pressure sensor 90 performs a series of discrete measurements and uses an algorithm to average these to obtain an accurate measurement of the weight of the vial 38. Some smoothing algorithms include moving average smoothing, rectangular or triangular un-weighted sliding average smoothing or other data smoothing algorithms know in the art. In an alternative embodiment, steps 204, 206 and 208 are performed at the same time as a single step.

In step 210, the processing unit 96 stores the image, bar code, text recognition, weight, and any other information input by the user into the fill apparatus 10 into the computer readable memory 98.

Steps 204-210 are also shown as a first vial step subgroup 211. This first vial step subgroup 211 comprises steps related to the determination and storage of information related to the vial 38.

In step 212, the user removes the vial 38 from the scale plate 86 and cleans the vial 38 using common aseptic techniques.

In step 213, the user places the vial 38 in the vial carriage 40. If a locking mechanism as described above is part of the fill apparatus 10 then this locking mechanism is engaged in this step.

In steps 214-220, a syringe 34 is placed on the scale plate 86 and in a similar manner to steps 204-210, the syringe 34 is placed, weighed, imaged and data associated with the syringe 34 stored in the computer readable memory 98. In addition to weight information on the syringe 34, the size of the syringe 34 may also be determined using image recognition algorithms like those known in the art for the measurement of lengths in images or may be entered into the system through keypad 18. Steps 214-220 may be included in a first syringe step subgroup 221 where the first syringe step subgroup 221 is comprised of steps related to the determination and storage of information related to syringe 34.

In step 222, the syringe 34 is placed in the syringe recess 16, ensuring the plunger 44 of the syringe 34 is securely placed in the plunger carriage 46.

In step 224, the user uses the keypad 18 to enter in the desired dosage. Optionally, the user may enter in information related to the size of the syringe 34. The processing unit 96 uses the information on dose and syringe 34 size and makes a calculation of the distance that the plunger 44 needs to move to load the required dose into syringe 34.

In step 226, the processing unit 96 commands the plunger motor 70 to rotate the plunger lead screw 68 to draw the plunger carriage 46 proximally to the distance required to pull an amount of air into the syringe 34 equal to the requested vial dosage.

In step 228, the processing unit 96 commands the vial motor 60 to rotate the vial lead screw 58 to move the vial carriage 40 such that the syringe needle 106 punctures the pierceable membrane of the vial 38 and continues to a point where the distal end of the syringe needle is entirely beyond the pierceable membrane.

In step 230, the processing unit 96 commands the plunger motor 70 to rotate the plunger lead screw 68 to move the plunger carriage 46 distally to the distance required to discharge all of the air in the syringe 34 into the vial 38. In an alternative embodiment, a motor torque or linear pressure sensor is incorporated into the system such that the processing unit 96 receives information from either of those sensors and senses an increase in the torque on the plunger motor 70 or the force on the plunger 44, indicating that the plunger 44 has been moved as far forward as is possible. Error handling may also be employed in the unit such that if the force or torque required to move the plunger 44 increases at a point contrary to where the processing unit 96 would expect these forces to increase, thereby indicating a blockage or other issue with the system, the system may alert the user to the condition and/or take steps to remediate the condition.

In an alternative embodiment, the torque on the vial motor 60 may be used to indicate the viscosity of the medication in the vial 38 and to adjust the fill parameters such as speed of fill based on this viscosity. In addition, the plunger carriage 46 may include force sensors which can determine the force on the plunger 44 and provide an indication of the viscosity of the medicine in the vial 38. By varying the speed of the plunger motor 70 and providing that information to a viscosity algorithm the precise viscosity of the medication can then be determined. Additionally, plunger motor 70 may be intermittently started and stopped and the force on plunger 44 determined to calculate whether the viscosity of the medication in the vial 38 is such that a negative pressure is created in the syringe 34 which can lead to bubble formation in the medication. The fill apparatus may then indicate to the user the potential bubble formation condition and alert the user to check the syringe 34 for bubbles. In addition, the calculated viscosity of the medication may be compared to a known viscosity for that medication to indicate to the user a potential issue with the medication or a potential misidentification of the medicine.

In step 232, the processing unit 96 commands the plunger motor 70 to rotate the plunger lead screw 68 to draw the plunger carriage 46 proximally to the distance required to pull an amount medicine into the syringe 34 equal to the requested vial dosage. In an alternative embodiment, the plunger carriage 46 may be drawn back further than the amount needed to pull the requested medicine amount into the syringe 34.

In Step 233 The fill apparatus can then vibrate either the plunger carriage 46 or the vial carriage 40 by rapid cycling of the corresponding carriage motor or other vibrational means know in the art. This moves any remaining air in the syringe 34 to the distal most portion of the syringe 34 where it can be removed by the user after the syringe is removed from the apparatus or the processing unit 96 may command to move the plunger motor 70 to rotate the plunger lead screw 68 to move the plunger carriage 46 distally to purge the air from the syringe 34 and provide the syringe 34 with the requested medicine dosage. In addition, a visual or conductive sensor (not shown) can be placed in the syringe recess 16 at the distal end of the cylinder portion 42. This sensor can be used to determine if all air has been purged from the syringe via image recognition algorithms, spectral analysis or other visual detection means known in the art.

Steps 234-240 and 242-248 repeat steps 204-210 and 214-222 respectively except that these steps are conducted after the filling of the syringe 34. Steps 234-240 may be included in a second vial step subgroup 241 where the second vial step subgroup 241 is comprised of steps related to the determination and storage of information related to vial 38. Steps 242-248 may be included in a second syringe step subgroup 249 where the second syringe step subgroup 249 is comprised of steps related to the determination and storage of information related to syringe 34.

In step 250 the weight of the vial 38 after the filling of the syringe 34 is compared to the weight of the vial prior to the filling of syringe 34. The processing unit 96 calculates the expected change in vial weight based on information associated with the medication, and the dosage filled in the syringe 34. The processing unit 96 then compares this expected weight change to the actual weight change calculated from the pre and post filling weighing steps. If the actual weight change and the expected weight change differ by more than a predetermined amount, the processing unit 96 indicates that an error has occurred and alerts the user via an audible tone and/or the display screen 20. The predetermined difference that indicates an error may be preset at manufacture of the fill apparatus or may be user selectable and settable.

In step 252, the comparison of step 250 is repeated for the syringe.

In step 254, the weight change of the syringe 34 is compared to the weight change of the vial 38 and the processing unit compares these two weigh changes against a predetermined difference. If the actual difference exceeds the predetermined difference, the processing unit 96 indicates that an error has occurred and alerts the user via an audible tone and/or the display screen 20. The predetermined difference that indicates an error may be preset at manufacture of the fill apparatus or may be user selectable and settable.

Steps 250-254 may be included in a comparison subgroup 255. Comparison subgroup comprises steps related to the comparison of weights of components obtain before filling the syringe 38 and after filling the syringe 38.

In optional step 256, the information obtained during the process is transmitted to an external medical records database like those described above. Optionally, the syringe fill apparatus 10, stores the information from a plurality of syringe fills in the computer readable memory 98 and transmits all the fill information to the external medical records database at one time. Alternatively, the syringe fill apparatus 10 stores the fill information on the removable memory device and the removeable memory device is removed from the syringe fill apparatus 10 and uploaded to the external medical records database by means of a computer operatively attached to the external medical records database.

It can be appreciated that the process flow detailed above is comprehensive and that one or more steps may be eliminated without preventing the other steps from being enacted. For instance, step 218 where the syringe 34 is weighed may be removed from the process flow in cases where the syringe 34 weight can be accurately determined form other information in the system. Also, the order of the steps may be changed where appropriate. For instance, the user may choose to execute steps 214-220 related to the syringe 34 before executing steps 204-210 related to the vial 38. Other step omissions and/or modifications are also contemplated.

In various embodiments, additional monitoring and control features may be provided on and/or in the syringe fill device. For example, the syringe fill device may comprise indicator lights, such as LED elements of differing colors, to indicate an operational state or condition of the syringe fill device. In one embodiment, LED elements include a red LED element that when energized indicates that the syringe fill device is not ready for syringe fill operation, and a green LED element that when energized indicates that the syringe fill device is ready for syringe fill operation. The syringe fill device may in various implementations comprise gauges, indicator lights, and/or other output features to provide a user with relevant information for the syringe fill process, such as, without limitation, temperature of fluid in the capsule(s), degree of completion of the fill operation, cumulative number of syringe fill operations performed in a specified time period, volume of the syringe being filled, etc.

The syringe fill device may also be provided with data communication and/or signal processing capability, e.g., a port for connection to a data communication or processing network, wireless connectivity to a fluid inventory monitoring system, etc. the syringe fill device may also be equipped with various input features and capability, e.g., settings mechanisms to accommodate a specific sized syringe, type of fluid, etc.

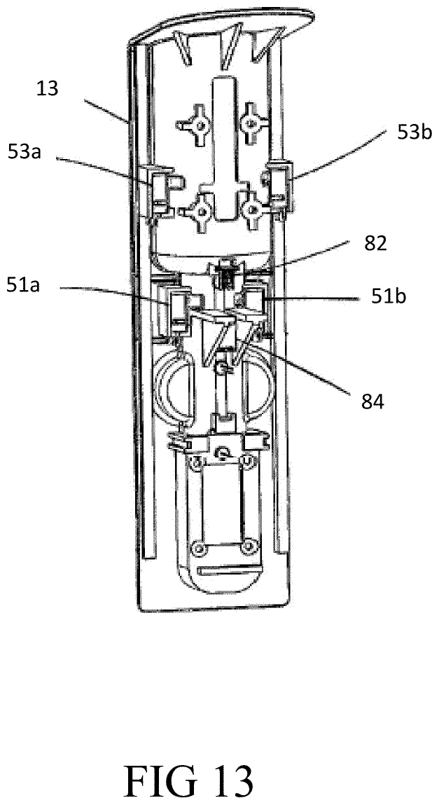

The syringe fill device additionally may be constructed with safety features, e.g., a “lock-out” feature such that the fill sequence is not able to be initiated unless a syringe is fully coupled with the cylinder fixator 48 and the vial is fully coupled to the vial carriage 40 to prevent initiation of fluid dispensing without a syringe and vial mounted to dispense the fluid. FIG. 13 shows a rear view of the recess housing 13. Mounted in recess housing 13 are syringe sensors 51a and 51b and vial sensors 53a and 53b. These sensors are placed such that they are on both sides of the vial recess 14 and the syringe recess 16. These sensors transmit and receive a light beam in either the visible or infrared spectrum. Insertion of the syringe 34 and the vial 38 break these beams to indicate proper insertion of the vial 38 and syringe 34 into the fill apparatus 10. Alternate sensor means are contemplated such as proximity sensors, switch sensors or contact sensors or any other position sensor known in the art. In addition, recess housing 13 contains a syringe temperature sensor 84 and a vial temperature sensor 82 for measuring the temperature of the respective syringe and vial placed in the fill apparatus 10. These sensors may be infrared temperature sensors, thermocouples, thermistors or other contact or non-contact temperature sensor known in the art.

Recess housing 13 may also include a sterilization lamp or lamps (not shown). The sterilization lamp or lamps are placed such that when the door 22 is closed the light from the lamps is sufficient to sterilize the vial and syringe in the vial recess 14 and the syringe recess 16. The lamps may emit UVA, UVB or UVC radiation or any other light spectrum capable of sterilizing non-porous surfaces. In an alternative embodiment, only the syringe recess is provided with light from a sterilization lamp. In another alternative embodiment the door further comprises a sensor (not shown) that indicates the position of the door 22 and prevents the sterilization lamps from activating until the door 22 is in the closed position.

The syringe fill device may also be constructed to incorporate in the interior volume of the housing a heating and/or cooling source such as an electrical resistance heating element a thermoelectric cooler, a fan or other heating or cooling systems known in the art, serving to maintain the fluids at temperature appropriate for physiological administration of the fluids. For example, the housing may contain a heater arranged to maintain such fluids at body temperature, to further enhance the administration of the composition comprising the fluid components.

It will be appreciated that the syringe fill device can further be provided with various monitoring and control elements and assemblies, to facilitate the syringe fill operation. For example, the syringe fill device may be arranged to fill 10 mL syringes, and the device may be equipped with visual and/or auditory output capability, to signal an operator when a syringe has been fully filled with 10 mL of therapeutic composition, thereby improving the speed and efficiency of the use of the fill apparatus 10. Additionally, the fill apparatus 10 may allow for input of syringe sizes and perform automated setup of the fill apparatus 10 specific to the size of the syringe to be filled, for instance the relative location of the vial carriage 40 to the cylinder fixator 48 and/or the relative distance from the plunger carriage 46 to the cylinder fixator 48 may be set based on the input information to improve the efficiency of the filling operation. In addition, the fill apparatus 10 may include a database of syringe types and manufacturers including pertinent measurement information on each type of syringe, allowing the use to select the syringe type and/or manufacturer and the setting appropriate fill settings in the fill apparatus 10 based on this information.

It will be also appreciated that the syringe fill device can further be automated by providing the computer readable memory 98 of the fill apparatus 10 with a database of information on known medications and patient dosing parameters such that the user may enter information on the patient, including but not limited to, age, sex and weight and the fill apparatus 10 would access the computer readable memory 98 and the processing unit 96 would determine the appropriate dosage to load into the syringe 34. In cases where dosing is automated, the fill apparatus 10 may also display the recommended dosage on the display screen 20 and also provide a request on the display screen 20 to confirm the recommended dosage prior to filling the syringe 34.

In addition, the fill apparatus 10 may include a physical data connection (not shown) such as an ethernet connection, an RS232 connection, a USB connection or other connection known in the art. This connection allows for the fill apparatus 10 to update its computer readable memory 98 or its firmware on the processing unit 96. These connections may also allow for connection of the fill apparatus 10 to a medical records system on a hospital network or to one that is cloud based. The fill apparatus 10 may also include wireless communication hardware to perform the communications tasks above. The connections may also permit an external processing unit such as a computer to be attached to the fill apparatus 10 for downloading data from the fill apparatus or uploading data to the fill apparatus 10. In an alternative embodiment, the fill apparatus 10 does not contain a processing unit 96 and instead, an external processing unit, such as a laptop computer is connected to the fill apparatus through the data connection and performs the processing functions of the fill apparatus 10.

Fill apparatus 10 may also be configured to mix medications. In this embodiment, the vial 38 and the syringe 34 are placed in the fill apparatus 10 as described for a simple medication fill operation. After the medication in the vial 38 is drawn into the syringe 34 and the vial 38 is moved away from the syringe needle 106, a second medication vial 38 is placed in the vial carriage 40 and the process is repeated, adding the second medication to the first in the syringe 34. In an alternative embodiment, the first vial 38 contains sterile saline or sterile water and the second vial 38 contains a dry medication to be hydrated. In this embodiment, the sterile saline is drawn into the syringe 34 and then, as described above the second vial 38 is placed in the vial carriage. When the second vial 38 is punctured by the syringe needle 106, the plunger 44 on the syringe 34 is advanced to transfer the sterile saline into the vial 38 to hydrate the dry medication. In an alternative embodiment of this process, the plunger 44 on the syringe 34 is repeatedly drawn back and pushed forward causing the saline/medication mixture to be transferred back and forth between the syringe 34 and the vial 38, aiding in mixing. As a final step, the mixed medicine is drawn into the syringe 34 at the prescribed dosage.

As a further alternative embodiment, the vial carriage 40 and/or the cylinder fixator 48 may include sensors for measuring the weight of the vial 38 and/or the syringe 34 such that the scale 24 is not required.

While the disclosure has been described herein in reference to specific aspects, features and illustrative embodiments, it will be appreciated that the utility of the disclosure is not thus limited, but rather extends to and encompasses numerous other variations, modifications and alternative embodiments, as will suggest themselves to those of ordinary skill in the field of the present disclosure, based on the description herein. Correspondingly, the invention as hereinafter claimed is intended to be broadly construed and interpreted, as including all such variations, modifications and alternative embodiments, within its spirit and scope.

I/We claim:

1. A syringe fill apparatus, comprising;
a scale;
an imaging device, comprising;
a camera; and
at least one imaging housing;
a vial carriage;
a vial carriage motor;
a plunger carriage;
a plunger carrier motor;
a cylinder fixator;
a processing unit; and
a computer readable memory,
wherein said imaging device is configured to capture an image an object, placed on said scale and wherein said object comprises at least one of;
a medicine vial; comprising;
a medicine receptacle;
a medicine; and
a pierceable membrane; and
a syringe, comprising;
a cylinder;
a plunger; and
a needle;
a patient record; and
a medicine record,
wherein said scale is configured to measure a weight of said object,
wherein said computer readable memory is configured to store said weight,
wherein said vial carriage is configured to moveably support said medicine vial, said plunger carriage is configured to moveably support said plunger and said cylinder fixator is configured to fixedly support said cylinder,
wherein said vial carriage motor is configured to move said vial carriage linearly along an axis of said medicine vial from a first vial position wherein said medicine vial is spaced apart from said needle to a second vial position wherein said needle pierces said pierceable membrane,
wherein said plunger carriage motor is configured to move said plunger carriage linearly along an axis of said plunger from a first plunger position wherein the plunger is disposed at an end of said cylinder to a second plunger position wherein the plunger is spaced away from said end of said cylinder,
wherein said movement from said first plunger position to said second plunger position draws said medicine into said syringe when said medicine vial is in said second vial position.

2. The syringe fill apparatus of claim 1, wherein said apparatus further comprises;
an input device;
a display device; and
a communication module,
wherein said processing unit is in communication with said input device and said display unit, wherein said input device is configured to accept an input entered by a user, said input comprising at least one of;
a medicine dosage input;
a medicine identifier input;
a patient information input;
a date input;
a syringe type input; and
a syringe size input,
wherein said input is communicated to said processing unit by said user input device and wherein said processing unit communicates said input to said display device to display said input on said display device and wherein said processing unit is configured to store said input in said computer readable memory as a data unit.

3. The syringe fill apparatus of claim 2, wherein said computer readable memory stores a plurality of data units.

4. The syringe fill device of claim 2, wherein said processor is configured to command said vial carriage motor to move said vial carriage from said first vial position to said second vial position and said plunger carriage motor to move said plunger carriage from said first plunger position to said second plunger position when said user enters said input.

5. The syringe fill apparatus of claim 4, wherein said input entered by said user is said medicine dosage and wherein said movement of said plunger from said first plunger position to said second plunger position draws into said syringe an amount of said medicine equal to said medicine dosage.

6. The syringe fill apparatus of claim 2, wherein said computer readable memory is configured to associate said plurality of said data units stored in said computer readable memory into a data record comprising at least two of;
said weight;
said image;
said medicine dosage input;
said date input;
said patient information input.

7. The syringe fill apparatus of claim 6 wherein said plurality of said data units stored in said computer readable memory are encrypted.

8. The syringe fill apparatus of claim 2, wherein said processing unit is configured to perform text recognition on said image to recognize a line of text on said object in said image, and wherein said line of text is stored in said computer readable memory.

9. The syringe fill apparatus of claim 6, wherein said data record further comprises said line of text.

10. The syringe fill apparatus of claim 6, wherein said syringe fill apparatus further comprises a bar code scanner,
wherein said barcode scanner is in communication with said processing unit,
wherein said bar code scanner is configured to read a barcode on said object and to communicate to said processing unit information contained in said barcode and wherein said processing unit is configured to store information contained in said barcode.

11. The syringe fill apparatus of claim 10, wherein said data record further comprises information contained in said barcode.

12. The syringe fill apparatus of claim 2, further comprising, a communications module,
wherein said communications module is configured to communicate with a database external to said syringe fill apparatus via a communication connection.

13. The syringe fill apparatus of claim 12, wherein said communication is one of, an ethernet connection, a USB connection, and RS232 connection and a wireless connection.

14. The syringe fill apparatus of claim 12, wherein said communications module encrypts communications with said database external to said syringe fill apparatus.

15. A method for filling a syringe, said method comprising the steps of;
activating a syringe fill apparatus;
placing a medicine vial on said scale;
obtaining a first vial information associated with said medicine vial;
placing a syringe on said scale;
obtaining a first syringe information related to said syringe;
placing said medicine vial in a vial carriage;
placing said syringe in a syringe fixator;
entering a dosage using a keypad;
processing said dosage using a processing unit;
sending commands from said processing unit to command a plunger carriage motor to retract a syringe plunger from a first plunger position wherein said syringe plunger is at the distal end of a syringe cylinder body to a second plunger position wherein said syringe plunger is retracted a sufficient distance to draw in a volume of air equivalent to a dosage;
sending commands from said processing unit to command a vial carriage motor to move a vial carriage from a first vial position wherein said vial is spaced apart from a syringe needle to a second vial position wherein said syringe needle pierces a pierceable membrane of said medicine vial;
sending commands from said processing unit to command said plunger carriage motor to move said plunger from said second plunger position to said first plunger position, injecting said air into said medicine vial;
sending commands from said processing unit to command said plunger carrier motor to retract said plunger carrier motor to retract said plunger to a third plunger position, wherein said third plunger position is greater than the distance necessary to draw in a volume of medicine equivalent to said dosage;
sending commands from said processing unit to command said vial carriage motor to move said medicine vial from said third vial position to said second vial position;
sending commands from said processor unit to command said plunger carrier motor to move said syringe plunger from said third plunger position to said second plunger position;
removing said syringe from said syringe fixator;
placing said syringe on said scale;
obtaining a second syringe information related to said syringe;
removing said medicine vial from said vial carriage;
placing said medicine vial on said scale; and
obtaining a second vial information related to said medicine vial.

16. The method of claim 15, wherein said step of obtaining said first vial information and said step of obtaining said second vial information further comprise;
obtaining a weight of said medicine vial;
obtaining an image of said medicine vial;
reading a barcode on said vial,
wherein said first vial information and said second vial information are then stored in a computer readable memory.

17. The method of claim 16, wherein said step of obtaining said first syringe information and said step of obtaining said second syringe information further comprise;
obtaining a weight of said syringe;
obtaining an image of said syringe;
reading a barcode on said syringe,
wherein said first syringe information and said second syringe information are then stored in said computer readable memory.

18. The method of claim 17, wherein the method further comprises;
a step of comparing said weight of said medicine vial obtained with said first vial information to said weight of said medicine vial obtained with said second vial information to obtain a vial weight difference and a step of comparing said weight of said syringe obtained with said first syringe information to said weight of said syringe obtained with said second syringe information to obtain a syringe weight difference.

19. The method of claim 18, further comprises;
A step of comparing said vial weight difference to said syringe weight difference to obtain a transfer difference and alerting a user if said transfer difference exceeds a predetermined value.

20. The method of claim 19, further comprising;
a step of transmitting said first vial information, said second vial information, said first syringe information, said second syringe information, said vial weight difference, said syringe weight difference, said transfer weight difference and a patient identifier to a medical records system.
